(19) United States
(12) Patent Application Publication
(10) Pub. No.: US 2008/0171355 A1
(43) Pub. Date: Jul. 17, 2008

(54) DEVICE FOR TESTING THE EFFECTIVENESS OF DISINFECTANTS ON PROTOZOAN PARASITES

(76) Inventor: ENG-HONG LEE, Goalph (CA)

Correspondence Address: SMART & BIGGAR;P.O. BOX 2999, STATION D, 900-55 METCALFE STREET, OTTAWA, ON, K1P5Y6, omitted

(21) Appl. No.: 11/939,182

(22) Filed: Nov. 13, 2007

Related U.S. Application Data
(60) Provisional application No. 60/868,998, filed on Dec. 7, 2006.

(51) Int. Cl. C12Q 1/22; C12M 1/00

(52) U.S. Cl. 435/31; 435/287.4

(57) ABSTRACT

This invention relates to a device for testing the efficacy of any disinfectant treatment, such as water disinfectants or equipment disinfectants. The device includes a culture of live unsporulated free-living protozoan parasites. If the disinfectant treatment successfully kills all or a substantial number of parasites, then the treatment is effective. One can determine whether the parasites have been killed or not by exposing them to sporulation conditions, and if they sporulate after exposure to the disinfectant treatment, then they were not killed by the disinfectant treatment.

This application claims priority from U.S. Provisional Application 60/868,998, entitled “Device for testing the effectiveness of disinfectants on protozoan parasites”, filed Dec. 7, 2006, the content of which is hereby incorporated by reference in its entirety.

### FIELD OF THE INVENTION

This invention relates to the field of testing the effectiveness of disinfectants, for example water disinfectants or equipment disinfectants.

### BACKGROUND

The following review of the background of the invention is merely provided to aid in the understanding of the present invention and neither it nor any of the references cited within it are admitted to be prior art to the present invention.

Water quality control companies, disinfectant companies, hospitals and others have a need for testing the effectiveness of disinfectants. There are no devices, methods or kits available in the market for testing the effectiveness of disinfectants against protozoan parasites. Yet, disinfectant properties are claimed for many chemical products and devices. Therefore, there is a need for a device, such as a kit, for testing the effectiveness of disinfectants.

### SUMMARY OF THE INVENTION

This invention relates to a device for testing the effectiveness of disinfectants. In one embodiment, the device is a simple “shake and incubate” device, such as a kit. In another embodiment, the device is adapted to test the effectiveness of disinfectants on free-living stages of protozoan parasites.

According to one aspect of the present invention, there is provided a device for testing the efficacy of a disinfectant treatment comprising (a) a culture of live unsporulated free-living protozoan parasites and (b) optionally, a receptacle for facilitating contacting of the disinfectant treatment with the culture.

According to another aspect of the present invention, there is provided a method for testing the efficacy of a disinfectant treatment comprising (a) contacting a culture of live unsporulated protozoan parasites in the free-living stage with an effective amount of the disinfectant treatment, and (b) determining the percentage of protozoan parasites that have survived, wherein the culture of (a) is stored under conditions that prevent sporulation prior to the contacting.

According to still another aspect of the present invention, there is provided a method for testing the efficacy of a disinfectant treatment comprising steps in the following order: (a) obtaining a stock culture of live protozoan parasite oocysts in the free-living stage containing a known and substantial quantity of live unsporulated oocysts and a known quantity of sporulated oocysts wherein the stock culture is stored under conditions that prevent sporulation; (b) determining the ratio of the sporulated oocysts to the total number of oocysts in the stock culture of oocysts; (c) exposing the stock culture of oocysts to the disinfectant treatment; (d) exposing the thus treated culture of oocysts to sporulation inducing conditions; (e) determining the number of unsporulated oocysts and sporulated oocysts in the treated culture; (f) determining the ratio of the sporulated oocysts to the total number of oocysts in the treated culture of oocysts; and (g) determining the effectiveness of the disinfectant treatment by comparing the ratio determined in step (b) with the ratio determined in step (f) and calculating the sporulation rate.

### BRIEF DESCRIPTION OF THE DRAWINGS

FIG. 1 is a drawing/picture of an unsporulated oocyst of Eimeria.

FIG. 2 is a drawing/picture of a sporulated oocyst of Eimeria.

FIG. 3 is a chart showing the deterioration rate of unsporulated culture of E. acervulina as predicted by the results obtained herein. The x axis provides the number of days that the culture is stored at 4° C. prior to exposure to sporulation conditions. The y axis provides the predicted percent sporulation after storage at 4° C. according to the x axis and following exposure to sporulation conditions, such as optimal sporulation conditions of 29.5° C. for about 21 to 24 hours.

### DETAILED DESCRIPTION OF THE INVENTION

There are many organisms, such as parasites, bacteria, fungi and viruses, that are present in drinking water and in the environment, such as on hospital equipment that are intended to be used on or in the human body, that may be hazardous to human health. Therefore, it is important to have efficacious disinfectants to kill such organisms in order to prevent human disease. A prime example of an organism that causes human disease and that may be acquired through contaminated drinking water is Cryptosporidium parvum. It is desirable to disinfect drinking water containing organisms such as Cryptosporidium parvum in order to prevent disease outbreaks.

The only living thing harder to kill than coccidian oocysts are prions. Therefore, if a chemical compound is shown to kill any of the free living stages of a protozoan parasite, especially that of Apicomplexa of which coccidian parasites are one example, such compound has been shown to be an effective disinfectant. That is, this disinfectant will be able to kill parasites, as well as bacteria, fungi and viruses.

Apicomplexa includes Coccidia (Cryptosporidium, Cyclospora, Isospora, Eimeria) and also includes Plasmodium, Babesia, Sarcocystis and Toxoplasma. The apicomplexa life cycle has three distinct life stages: sporogony, merogony and gametogony. Merogony, in which merozoites are formed, and gametogony, in which gametes are formed, occur inside the host, and are not the free living stage of a protozoan parasite. Sporogony occurs immediately after a sexual phase and is an asexual reproduction or cell division that culminates in the production of sporozoites. Oocysts are the free-living stage of a protozoan parasite. Oocysts can be shed in the unsporulated form (Eimeria, Cyclospora) or in the sporulated form (Cryptosporidium, Isospora). When sporozoites are visible inside the oocysts, this indicates that sporulation has occurred. Typically, when an Eimeria oocyst is passed in the feces, it is not infective because it does not contain sporozoites; this is an unsporulated oocyst. Sporozoite formation, or sporulation may be stimulated when the oocyst is exposed to oxygen, and hence when the oocyst is outside the body of its host. A host is infected when it ingests sporulated oocysts that have been previously passed in the feces of another host. The oocyst excysts in the host's small intestine, and the sporozoites contained within the oocyst are liberated. The sporozoites penetrate the cells of the host's small intestine and reproduce asexually.

Cryptosporidium parvum is an example of a coccidian parasite that has been occasionally identified in surface waters throughout Canada (Roach et al., 1993; Wallis et al., 1996; Isaac-Renton et al., 1999). Several outbreaks have occurred through ingestion of this parasite when municipal drinking water supplies from surface sources were not effectively treated (Hrudey and Hrudey, 2004). Similarly, therefore, if campers or hikers ingest water directly from streams, reservoirs, or rivers, they too might become ill from this parasite. This is partly supported by self reporting surveys from long distance backpackers which concluded that the risk of diarrhea is greater among those who frequently drink untreated water from streams or ponds (Boulware et al., 2003).

The main challenge in controlling coccidian parasites such as Crytosporidium is that the oocyst, or infective stage, is extremely resistant to chemical disinfection (Quinn and Markey, 2000), purportedly because of its thick lipid layer similar to the Eimerian oocyst wall (Stotish et al., 1978). Most strategies for control rely on encapture of oocysts by filtration. Nevertheless, some purification tablets or devices sold in camping equipment stores make label claims that their products can inactivate Cryptosporidium. The devices, kits, assays, methods and uses of the invention may be used to test such claims.

Because Cryptosporidium is a human parasite, a related coccidian parasite, Eimeria acervulina, a parasite of chicken, may be used as a surrogate. Chicken Eimeria is safe for humans to handle, more economical to work with and has been used successfully as a surrogate in other published studies (Lee and Lee, 2001; 2003).

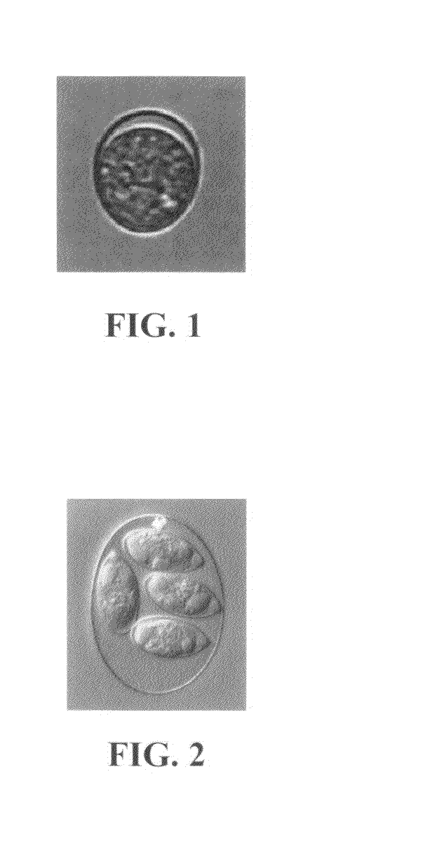

This invention is a useful way of testing disinfectants using unsporulated live oocysts of Eimeria. Unsporulated Eimeria oocysts containing one lump sporoplast are morphologically distinct from sporulated occysts containing 4 spindle-shaped sporocysts. This can clearly be seen in FIG. 1, which shows an unsporulated Eimeria oocyst and is in contrast with FIG. 2, which shows a sporulated Eimeria oocyst. The efficacy of a disinfectant can be determined in a short time or overnight by testing with unsporulated Eimeria oocysts. With conventional infectivity testing, the effectiveness of a disinfectant on a sample product containing organisms would be tested by passing the sample product through a host and collecting the organisms through the host. However, this creates a “blackbox effect”, meaning that the immune response of the host may kill off some of the organisms or allow the organisms to reproduce, and it is impossible to tell how many organisms the disinfectant killed. With the testing of this invention, in comparison with infectivity testing, there is no interference of the “blackbox effect” of infection by having the parasite pass through the host for several days and involving the immune response of the host to determine disinfectant efficacy. With Eimeria, the effectiveness of disinfectants can be proportionally analysed via the sporulation rates. In other words, the Eimeria oocyst will not multiply outside the host, therefore the numbers of oocysts are constant and are easy to keep count of.

Very few parasites can be maintained for any length of time in the unsporulated form outside the host body. Cryptosporidium, for example, is shed in a sporulated form or when it is immediately outside of the host body.

In contrast, Eimeria, which is a genus of chicken coccidia, are unsporulated when they initially leave the body of the chicken. Oxygen exposure will cause the chicken coccidia to sporulate at a certain rate.

In contrast to Cryptosporidium, Eimeria are easier to collect and safe for human handling. Eimeria species are particularly infective in birds, producing lesions in young birds even at low doses. Furthermore, one can test the survival of Eimeria without the necessity of testing through infectivity. Infectivity testing means that one must infect an animal, such as a mouse, with a sample of material disinfected by a disinfectant and determine the effectiveness of the disinfectant by counting the parasites that survive in the animal. Infectivity testing is inaccurate because it depends in part on the host's ability or inability to fight off infection and the number of parasites in the host could increase due to parasite reproduction within the host. Furthermore, testing Cryptosporidium liveability involves a process called excystation of oocysts, which can only be performed by a highly skilled person or laboratory. By testing unsporulated Eimeria, one can test their survival merely by looking at the sample of material disinfected by a disinfectant through a microscope to determine whether the Eimeria have sporulated or not. If the unsporulated Eimeria are killed by the disinfectant, then they will not sporulate because they are dead. If the unsporulated Eimeria survive the disinfectant they will sporulate. By counting the number of sporulated Eimeria versus the number of unsporulated Eimeria, 1) before exposure to the disinfectant and 2) after exposure to the disinfectant and following exposure to sporulation conditions, one can determine the effectiveness of the disinfectant. Testing disinfectants on unsporulated Eimeria in vitro is more accurate than any infectivity testing. That is, Eimeria will not multiply outside the host and therefore the numbers of Eimeria oocysts in a sample will remain constant. It is only the oocysts' life cycle stage which may be altered outside of the host, from unsporulated to sporulated. An unsporulated oocyst will convert from unsporulated to sporulated only if it survives exposure to the disinfectant.

Coccidian parasites of chicken are generally safe for humans to handle. There are coccidian parasites that infect humans, such as Cyclospora. However in one embodiment of the invention, coccidian parasites that do not infect humans are used. In another embodiment, the coccidian parasites used are Eimeria. In particular, in one embodiment of the invention, the coccidian parasites used are Eimeria acervulina. In one embodiment of the present invention, unsporulated oocysts provided in a device, such as a kit, for testing the effectiveness of disinfectants are stored for 1 day, 1 week, 2 weeks, 3 weeks, one month or more than one month.

In general, the devices, methods and kits of the invention should be refrigerated to prevent sporulation prior to use. In one embodiment, the refrigeration temperature for storing the unsporulated free-living protozoan parasite is from 2° C. to 8° C. Persons skilled in the art will know that stating that a refrigeration temperature of 4° C., means that the refrigeration temperature may range between 2° C. to 8° C.

In another embodiment, the unsporulated oocysts are stored at about 2° C. In another embodiment, the unsporulated oocysts are stored at about 3° C. In another embodiment, the unsporulated oocysts are stored at about 4° C. In another embodiment, the unsporulated oocysts are stored at about 5° C. In another embodiment, the unsporulated oocysts are stored at about 6° C. In another embodiment, the unsporulated oocysts are stored at about 7° C. In another embodiment, the unsporulated oocysts are stored at 8° C.

A “disinfectant treatment” is any treatment that is believed by anyone or advertised to have disinfectant properties. Disinfectant treatments include but are not limited to heat, UV light, bleach (Cl₂), peracetic acid, iodine, chlorine dioxide, oxidants, and mixed oxidants.

Disinfectant treatments that may be tested with the devices or methods of the invention include treatments that currently exist and importantly include treatments that have yet to be developed. The devices or methods of the invention will help those in need to test the efficacy of both new and old disinfectant treatments and provide a way to compare the efficacy of various disinfectant treatments.

Disinfectant treatments may be administered in a variety of ways, such as through devices, chemical compounds and compositions etc. Disinfectant treatments that are compositions may comprise a chemical compound together with an excipient.

All patents, patent applications and publications mentioned herein, both supra and infra, are hereby incorporated by reference in their entirety.

While the invention has been described with reference to certain specific embodiments and will be described in the following Examples, it is understood that it is not to be so limited since alterations and changes may be made therein which are within the full and intended scope of the appended claims.

Now in order to more particularly define some embodiments of the present invention, the following Examples provide details of devices, kits, assays, methods and uses of the invention.

### EXAMPLE 1

### Collection, Storage and Use of Eimeria acervulina

Introduction: Eimeria acervulina oocysts have been used as a surrogate for human coccidia such as Cryptosporidium and Cyclospora, for studies related to public health where both sporulated and unsporulated oocysts were used as test organisms (Lee & Lee, 2001, 2003). However, some of the unsporulated oocysts collected over night had sporulated. Even though the sporulation rate reported was low, it may make collection of clear evidence difficult (Lee & Lee, 2003). Therefore, the following methods of collection, storage and use of these unsporulated E. acervulina oocysts may improve their use as a test organism for testing disinfectants. When unsporulated oocysts were prepared in this manner, they were shown to have sporulated at the maximum rate for at least one week; they can act as test organism even at declined rates a month later; besides providing result overnight, sporulation rates may be used to calibrate the capacity of inactivation of the test disinfectants.

Oocyst collection: Unsporulated oocysts of E. acervulina were harvested and used from duodenal scrapings from chicks 5 days after inoculation. They were disinfected and kept at 4° C. until use.

Sporulation: After exposing these unsporulated oocysts to treatments, oocysts were sporulated in 2.5% potassium dichromate, at 29.5° C., for a minimum of 20 hours. Oocysts after sporulation appear as 4 well defined sporocysts within each oocyst, very distinct from the undifferentiated or unsporulated oocysts.

Counting: Oocysts were counted in a haemocytometer under the microscope. At least 250 sporulated or unsporulated oocysts were counted for each determination of sporulation rate.

### EXAMPLE 2

### Characterizing Sporulation rates of Eimeria acervulina Under Varying Conditions

Effects of Storage at 4° C. on Sporulation Rate of Eimeria acervulina

Oocysts of Eimeria acervulina were harvested from chickens. Some oocysts were immediately sporulated in potassium dichromate (standard procedure in this art) and kept cold while being transported over about 2 hours.

An aliquot was sporulated overnight (fresh) in potassium dichromate and the rest stored at 4° C. for 4 days (96 hours) before sporulation.

The tables below list the storage conditions of oocysts of Eimeria acervulina and the number of sporulated and unsporulated oocysts. To determine the presence of sporulation, the oocysts were subjected to examination under a microscope.

TABLE 1
Sporulation of E. acervulina following storage for 2 hours
at 4° C. followed by incubation at 17.5 hrs at 30° C.

Control
19.5 hrs. at 4° C.
(0 hrs. incubated)

| | |
|---|---|
| 2 Hrs. at 4° C. | Unspr. (number of |
| (17.5 hr. at 30° C.) | defective oocysts |

| Spr. | Unspr. | Spr. | in brackets) |
|---|---|---|---|
| 27 | 12 | 0 | 126 (21) |
| 23 | 16 | 1 | 54 (12) |
| 25 | 11 | 0 | 59 (9) |
| 20 | 10 | 0 | 110 (20) |
| 106 | 41 | Total: 1 | Total: 349 (62) |

| | | |
|---|---|---|
| Total: 201 | Total: 90 | 349/411 (84.9%) |
| | | percent of unsporulated |
| | | oocysts that are not |
| | | defective is 84.9% |

201/291 = 69%
69% ÷ 84.9% = 81.3%

In Table 1, among the unsporulated controls there were about 15% that would not sporulate because of deformity or emptiness inside the oocysts or very little substance inside (defective oocysts). Therefore, only about 84.9% of oocysts appeared to be viable for sporulation. Therefore, 84.9% is the correction factor which takes into account the defective or damaged oocysts.

The actual counts were made and resulted in 69%, and should be corrected by dividing it by 84.9%, for a corrected count of 81.3%.

It can be seen from the control group that only 1 of 412 was counted as sporulated, or the level of sporulation was 0.24% prior to incubation, and taking into account the factor of 84.9% of viable/non-damaged oocysts, the level of sporulation was 0.28%.

Each row in Table 1 represents the count of sporulated or unsporulated oocysts per sample placed under a slide or per region for a larger sample and counted under a microscope.

TABLE 2
Sporulation of E. acervulina following incubation at 24 hrs at 29.5° C.
2 hrs at 4° C.
24 hrs at 29.5° C.

| Spr. | Unspr. |
|---|---|
| 88 | 45 |
| 139 | 48 |
| 138 | 53 |
| 365 | 146/511 |

365/511 ≡ 71.4%
Corrected
71.4%/.849 ≡ 84.1%

### Conclusion:

Freshly collected, E. acervulina have sporulated at about 70% (69+71) and 82.5% (81%+84%) after correction.

Oocysts were collected from chickens and were kept overnight (about 18 to 20 hours) in 2.5% Potassium dichromate at room temp (23° C.) from 4° C. 120 hrs.

TABLE 3
Sporulation of E. acervulina following storage for 120 hours (5 days)
at 4° C., followed by incubation for 18 to 20 hours at 23° C.
23° C. overnight (room temperature) on the bench (120 hrs at 4° C.)

| Spr. | Unspr. |
|---|---|
| 6 + 3 | 18 |
| 4 | 10 |
| 3 + 1 + 1 | 11 + 2 |
| 4 | 2 |
| 4 | 10 |
| 26 | 53 |

26/79 = 32.9%

Incubating at 29.5° C. appears to be the most optimum temperature for sporulation. However, the oocysts of this invention may be sporulated using any of a variety of conventional methods. One of skill in the art may select the most appropriate sporulation techniques without departing from the scope of this invention.

TABLE 4
Sporulation rate of E. acervulina following storage for 96 hrs. at 4°
C., followed by incubation for 21 hrs at 29.5° C. 96 hrs. at 4° C.
21 hrs at 29.5° C.

| Spr. | Unspr. |
|---|---|
| 58 | 24 |
| 67 | 27 |
| 46 | 23 |
| 42 | 30 |
| 61 | 27 |
| 274 | 131 |

274/405 = 67.6%
67.6% ÷ 84.9% = 79.6% (−4.5%)
A loss of about 4 to 5% after 4 days at 4° C.

Compare this result with 120 hrs at 4° C. and not sporulated (see Table 5).

TABLE 5
Minimal sporulation of E. acervulina following storage for 120 hrs. at
4° C., and not sporulated. Note: Oocysts taken from culture bottle
120 hrs at 4° C. (Not sporulated)

| Spr. | Unspr. | Damaged |
|---|---|---|
| 1 | 47 | 15 |
| 0 | 40 | 9 |
| 0 | 16 | 3 |
| 0 | 10 | 4 |
| 0 | 17 | 2 |
| 0 | 18 | 2 |
| 0 | 15 | 7 |
| 0 | 25 | 3 |
| 0 | 19 | 4 |
| 1 | 207 | 49 |

207/256 = 80.85%
207/257 = 80.54%
84.9% − 80.85% = 4.05%
84.9% − 80.54% = 4.36%
The loss is found on the damaged unspr. oocysts.

TABLE 6
Summary of data showing sporulation rates of
E. acervulina under varying storage conditions.

| | | % Sporulated | % Unsporulated |
|---|---|---|---|
| At 4° C. | Incubated | Oocysts (Spr.) | oocysts |
| 2 hrs. | 17.5 hrs at | 69% | 31% |
| | at 30° C. | (from Table 2) | (from Table 2 |
| 96 hrs. | 21 hrs at | 67.7% | 32.3% |
| | 29.5° C. | (from Table 4) | (from Table 4) |

TABLE 7
No sporulation of E. acervulina following
storage for 11 days at 4° C.

| 11 days at 4° C. No Sporulation | | | |
|---|---|---|---|
| Spr. | Unspr. | Damaged | Total Count |
| 0 | 244 | 45 | 2.9 × 10⁶ |

45/289 = 15.5% damaged
Not much different from the count of first day Jul. 22, 2005

The above Table shows that the shelf life of the unsporulated oocyst can be at least 11 days.

TABLE 8
Sporulation of E. acervulina following storage for 11 days
at 4° C., followed by incubation for 22 hrs at 30° C.
11 days at 4° C./30° C. for 22 hours

| Spr. | Unspr. | Damaged |
|---|---|---|
| 19 | 69 | 20 |
| 23 | 74 | |
| 26 | 76 | |
| 34 + 2 | 80 | |
| 24 | 72 | |
| 18 | 82 | |
| 30 | 71 | |
| 27 | 79 | |
| 27 | 70 | |
| 230 | 693 | |

230/923 = 24.9%
A large decline in sporulation rate in 11 day old oocysts

It can be seen from Tables 1, 5 and 7 that there are few sporulated oocysts of E. acervulina present where the oocysts are fresh, meaning that they have been incubated for 0 hours (not incubated), or stored at 4° C. Table 1 shows that only 0.24% of oocysts were sporulated at 0 hours incubation, or 0.28% were sporulated when adjusting for damaged oocysts by a factor of 84.9%. Table 5 shows that at 120 hours at 4° C., only 0.39% of oocysts were sporulated, or 0.46% when adjusting for damaged oocysts by a factor of 84.9%. Table 7 shows that after 11 days at 4° C. no oocysts were sporulated.

From the above results, it is clear that any oocysts that have a “good” or viable appearance, will sporulate.

That is the sporulation rate can be predetermined by counting the oocysts in stored culture. If one counts the “good” oocysts and corrects for the count of “damaged” oocysts, one can obtain a corrected percentage of “good” oocysts from freshly sporulated samples.

Alternatively, one may simply, obtain the balance of damaged oocysts, deduct the difference between sporulated from non-sporulated and use the difference as the constant. Deduct this constant from the percent of the “good” oocysts.

For example,

at 0 hr at 4° C./18 hr. 29.5° C. (18 hrs minimum required time).

From Table 1: the % unsporulated oocysts where the damaged oocysts have been deducted at 0 hr at 4° C. is 84.9%.

From Table 2: the % sporulation following sporulation conditions of 18 hrs at 29° C. is 71.4.

The constant is calculated as follows:

84.9%−71.4=13.5%

Therefore, 13.5 is the constant. The constant is the percent of unsporulated oocysts that may appear to be intact but do not sporulate.

The predicted % “good” unsporulated oocysts after 120 hrs at 4° C. was 80.85%. (See Table 5 where the number of unsporulated oocysts that appear to be viable under the microscope is 80.85%).

The predicted % sporulation following incubation/sporulation conditions is:

80.85%−13.5%=67.35%.

Actual % sporulation was =67.6% (at 96 hrs 4° C.) (see Table 4) or roughly 1%/day. That is the number of viable unsporulated oocysts decreased by about 1% per day.

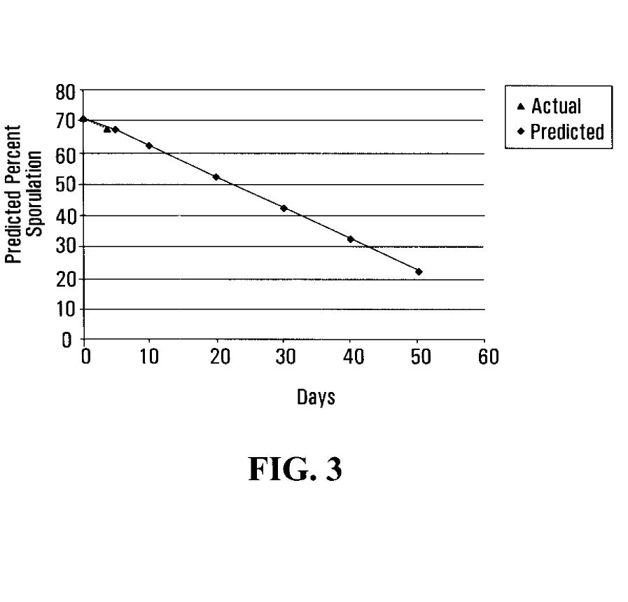

If the predicted and actual sporulation rates stay consistent, it means that the unsporulated culture of E. acervulina can be used as a useful surrogate if the rate of deterioration is predictable. FIG. 3 provides an example of a prediction of the rate of deterioration of an unsporulated culture of E. acervulina.

It will not be a surprise if most of the sporulation rates are predictable; samples obtained can be very useful for any test or for disinfection tests. If unsporulated oocysts, that is fresh oocysts or oocysts stored at 4° C., are exposed to a disinfectant, only those oocysts that survive will sporulate, and by counting the number of sporulated oocysts, one can determine the effectiveness of the disinfectant.

Moreover, if the loss of unsporulated oocysts is 1% a day, then we know that if the starting sporulation rate is 71.9%, for example, this culture will be good for at least 21 days (3 weeks) to get to 50% or about 40 days to get to ⅓ sporulation rate or this culture will be good for at least 1 month or more, if stored at 4° C. In other words, there will be a sufficient number of unsporulated oocysts present in a test device, kit or method of the invention to test a disinfectant after storing the freshly collected oocysts at 4° C. for at least 1 month or more.

### EXAMPLE 3

### Testing the Effectiveness of Chlorine as a Disinfectant on Oocysts

Tables 9 and 10. Sporulation of E. acervulina following storage for 144 hours at 4° C., followed by incubation for 20 hrs at 29.5° C., followed by application of 25,000 ppm Cl₂, 2,500 ppm Cl₂ or 0 ppm Cl₂

TABLE 9
144 hrs at 4° C. 20 hrs at 29.5° C.

| | |
|---|---|
| 25,000 ppm Cl₂ | 2,500 ppm Cl₂ |

| Spr. | Unspr. | Spr. | Unspr. |
|---|---|---|---|
| 3^{p} | 23 | 15^{p} | 20 |
| 3^{p} | 30 | 12^{p} | 20 |
| 4^{p} | 18 | 7^{p} | 18 |
| 8^{p} | 26 | 12^{p} | 29 |
| 6^{p} | 32 | 12^{p} | 21 |
| 1^{p} | 44 | 10^{p} | 14 |
| 9^{p} | 32 | 22^{p} | 49 |
| 7^{p} | 18 | 15^{p} | 38 |
| 8^{p} | 34 | 18^{p} | 37 |
| 49 | 257 | 123 | 246 |

49/306 = 16% Sporocyst Stage only
123/369 = 33.3% Sporocyst stage only
^{P}-sporocysts only (some damaged)

TABLE 10
Control
144 hrs at 4° C.
20 hrs at 29.5° C.
0 ppm Cl₂

| Spr. | Unspr. |
|---|---|
| 32 | 10 |
| 29 | 13 |
| 21 | 7 |
| 23 | 11 |
| 23 | 11 |
| 16 | 6 |
| 15 | 10 |
| 22 | 10 |
| 20 | 9 |
| 201 | 87 |

201/288 = 69.8%

### Conclusion:

To date, the unsporulated oocysts can be used for at least up to 6 days at 4° C. with very little loss in efficacy.

Chlorine at ˜25,000 ppm was highly effective in inhibiting sporulation. Chlorine at 2,500 ppm was also highly effective.

The minimum effective suppression level of Cl₂ on sporulation rate of E. acervulina is between 1,000 ppm and 2,500 ppm.

Infectivity testing is laborious and inaccurate. The problem is how to distinguish between the sporocyst non-infective stage and the infective stage other than by infectivity. This may be accomplished by distinguishing the spindle-shape sporocysts (infective) from round-shape sporocysts (non-infective).

If one counts only the spindle-shape sporocysts, then one is counting only the infective sporulated oocysts.

The lack of spindle-shape sporocysts indicates that a disinfectant was effective after its application to the oocysts.

Samples left at Room Temperature (23° C.) were well sporulated.

By the time the 4-sporocyst stage have the 2-spindle-shaped sporozoites in each sporocyst; the oocysts have already gone through the aerobic stage of differentiation. In other words, disinfectants that cannot prevent this development are not effective.

After the formation of sporozoites, if they are not completely mature, they contain almost homogeneous particles floating around but once matured, they are well structured with 4-spindle-shaped sporozoites (FIG. 2).

### EXAMPLE 4

### Testing the Effectiveness of UV light as a Disinfectant on Oocysts

TABLE 11
Sporulation of E. acervulina following storage for 12 days at 4° C.,
followed by incubation for 18 hrs at 30° C., followed by either
application of UV Treatment or no UV Treatment

| | |
|---|---|
| UV Treated | Control (no UV treatment) |
| 12 days at 4° C. | 12 days at 4° C. |
| 18 hrs. at 30° C. | 18 hrs at 30° C. |

| Spr. | Unspr. | Spr. | Unspr. |
|---|---|---|---|
| | 28 | 11 | 40 |
| 0 | 64 | 17 | 38 |
| 1 | 72 | 13 + 2 | 36 |
| 0 | 82 | 17 | 29 |
| 0 | 60 | 17 | 40 |
| 1 | 306 | 11 | 27 |
| | | 11 | 42 |
| 0.3% | | 16 | 38 |
| | | 14 | 44 |
| | | 129 | 334 |

129/463 27.8%

UV light device appeared to work as a disinfectant because only 0.3% of oocysts were able to sporulate after storage for 12 days at 4° C. followed by exposure to the UV light device and then followed by incubation/sporulation conditions of 18 hrs at 30° C.

Unsporulated oocysts that were kept at 4° C. for 12 days and were not given UV treatment (control) still gave a sporulation rate of 27.8%. This means that at least 27.8% of the oocysts were still viable after storage at 4° C. for 12 days because they were able to sporulate after exposure to sporulation conditions of 18 hrs at 30° C.

All of the groups 1 to 6 have 5 chicks per group. These 6 treatment groups were: 1. 4 days at 4° C. and sporulated at 29.5° C. for 21 hours; 2. 6 days at 4° C. and sporulated at 29.5° C. for 20 hours; 3. chlorine treated 25,000 ppm; 4. chlorine treated 2,500 ppm; 5. 12 days at 4° C. and sporulated at 30° C. for 18 hours; and 6. UV treated. All of the birds in all groups were infected after the percent sporulation data in Table 12 were collected to test infectivity. The dose of Elmeria acervulina oocysts given to each bird was adjusted to 133 RD of Immucox™ for 4 day sample (Group 1). “RD” means “recommended dose”, which is 2/15^{th} of a vial of Immucox™.

Birds in groups 3, 4 and 6 were all kept in one box. Birds in groups 1, 2 and 5 were kept in another box.

TABLE 12
Birds tested for infectivity

| | 5 birds/22 hrs/~36 gm feces | | |
|---|---|---|---|
| | collected 5 days post innoculation | | |
| Group | All pooled | | 100× Magnification |
| 6 | +ve | Numerous | (~25/field) |
| 4 | +ve | Numerous | (~100/field) |
| 3 | +ve | Few | (~6/field) |
| 5 | +ve | Numerous | (~200/field) |
| 2 | +ve | Numerous | (~200/field) |
| 1 | +ve | Numerous | (~200/field) |

25,000 ppm Cl₂

TABLE 13
Comparison of unsporulated oocysts obtained from scrapings
at Day 5 and those collected between Day 5 and Day 6.
Unsporulated oocysts from scrapings collected 24 hrs (28° C.)

| | |
|---|---|
| Day 5 | Day 5-6 |

| Spr. | Unspr. | Spr. | Unspr. |
|---|---|---|---|
| 0 | 266 | 10 | 241 |

10/251 = 3.9%

TABLE 14
Sporulated oocysts of E. acervulina after fresh scrapings
from chicken intestines are incubated overnight at
29.5° C., followed by application of 25,000 ppm
Cl₂ 25,000 ppm Cl₂
Scraping

| Spr. | Unspr. |
|---|---|
| 119 | 30 |
| 115 | 37 |
| 133 | 42 |
| 367 | 109 |

367/476 = 77.1%

TABLE 15
Sporulated oocysts of E. acervulina after fecal
collection from chicken are kept overnight
Fecal collection (overnight 22 hours)

| Spr. | Unspr. |
|---|---|
| 48 | 25 |
| 34 | 20 |
| 20 | 16 |
| 30 | 15 |
| 23 | 13 |
| 155 | 89 |

155/244 = 63.5%

There is no less sporulation in a scraping from chicken intestine than in the chicken fecal collection.

TABLE 16
Effect of Chemical Disinfectants and UV Light on the Sporulation
Rate of Oocysts of Eimeria acervulina, a Chicken Coccidium

| | Disinfectant/ | Incubate at | Sporulation * |
|---|---|---|---|
| Treatment | Disinfection | 30° C. (20 hrs) | Rate (%) |
| Control, Positive | None | yes | 76.1 |
| Chlorine | 2.5% 2 hours | yes | 59.3 |
| | (20° C.) | | |
| Peracetic Acid | 1% 2 hours | yes | 65.9 |
| | (20° C.) | | |
| UV (SteriPen) | 32 oz. | yes | 5.5 |
| Control, Negative | None | no | 6.1 |
| (6 days at 4° C.) | | | |
| Control, Negative | None | No | 3.9 |
| (fresh) | | | |

* At least 250 oocysts were counted.

All unsporulated oocysts were harvested from feces collected from infected day-old chicks 5 days post inoculation. Collection was made in 24 hours.

### EXAMPLE 5

### Devices, Kits and Methods for Disinfectant Testing

An embodiment of a device for disinfectant testing includes the following:

A bottle of unsporulated E. acervulina culture;

A specification sheet on sporulation rate of this culture;

A stack of 5 oz. (150 ml) wide-mouth screw-cap plastic containers.

One volume of culture and a test volume of a test disinfectant;

In one embodiment, a device for disinfectant testing includes the following instructions:

Let the treated sample sit for a 4 hours or recommended amount of time required for disinfection;

Shake well and let it sit overnight;

Count the rate of sporulation using a microscope and compare that with the stored culture;

Alternatively, let the treated sample sit for 4 hours or recommended amount of time required for disinfection;

Shake well and return it to a laboratory by courier, such that the laboratory staff will count the rate of sporulation using a microscope and compare that with the stored culture;

In another embodiment, a device of the invention includes the following:

1 ml of culture in small (5 oz) plastic jar+10 ml of disinfectant;

instructions to pour the disinfectant into the plastic jar of culture;

cap the plastic jar, shake the jar for a minute and incubate at 30° C. overnight.

Shake well, take a drop of the mixture and put it on a slide and put it under the microscope and count and determine the rate of sporulation. Use caution if testing caustic or corrosive materials.

### EXAMPLE 6

### Testing Water Purification Chemicals and Treatment Device for Campers and Hikers Using Unsporulated Oocysts of Eimeria acervulina

In Example 6, a number of water disinfection products were tested using unsporulated oocysts of Eimeria acervulina. Iodine tablets have been shown to be ineffective in inactivating Cryptosporidium (Gerba et al., 1997). They were tested again here because one iodine package label read “not tested against Cryptosporidium”. Contact times of 40 minutes for Coghlan's Drinking Water Tablets and 30 minutes for the Polar Pure resulted in sporulation rates of 66% and 68%, respectively (Table 17) showing no significant lowering than the positive control at 73%. With fewer oocysts, the iodine products were marginally better at 50% and 62%, respectively (Table 18), but again not significantly better than the controls. Overall, the iodine products, similar to the previous study (Gerba et al., 1997), were not effective in inactivating coccidian parasites.

One of the chlorine dioxide products, Micropur MP 1 tablets, had a contact time of 4 hours as recommended by the manufacturer to kill “Cryptosporidium and Giardia” while the other, Pristine tablets, had a contact time of 30 minutes and a recommended triple dose as recommended by the manufacturer “to treat for Cryptosporidium”. Nonetheless, the sporulation rates for the chlorine dioxide were 78% and 73%, respectively, when a higher number of oocysts were used as spikes (Table 17), which were not significantly different from the positive controls (73%). When lower numbers of oocysts were treated with chlorine dioxide products (Table 18), their sporulation rates were 18% and 26%, respectively. However, neither chlorine dioxide product could completely inactivate coccidian oocysts.

The MIOX® is a device in which an electric current runs through primarily a brine solution and likely other chemicals to create mixed oxidants of free chlorine, chlorine dioxide, hydrogen peroxide, ozone, and other short-lived (unspecified) oxidants (Venczel et al., 1997). These oxidants are then immediately added to water as treatment. Following the manufacturer's recommendation, “treating water contaminated with Cryptosporidium requires a 4-hour total wait time”, this system was not completely effective in inactivating oocysts of Eimeria acervulina with sporulation rates around 50%, even with the lower number of oocysts in the challenge.

The SteriPEN™, a hand held battery operated device, appeared to provide the most effective treatment tested in Example 6 whether the oocysts treated were at high (Table 17) or at low levels (Table 18) and turbidity likely did not play a role in these treatments. These results confirmed the claim made by the manufacturer that ultraviolet light can destroy Cryptosporidium in clear water and Example 6, using Elmeria, supports such claim.

The highest concentration of Cryptosporidium oocysts so far documented in a surface water source was about 30,000 oocysts/100 L (Report of the Commission of Inquiry into matters relating to the safety of the public drinking water in the City of North Battleford, Saskatchewan, 2002). To facilitate recovery and counting, however, we have introduced undoubtedly a high challenge in the number of oocysts into Example 6, which may not be representative of what a camper or hiker may encounter. However, when treatment methods were effective, such as by heating or ultraviolet light, the same treated numbers 2×10⁶ and 1×10⁵ oocyts/250 mL like others (Tables 17 and 18) were inactivated at the same rate as the heated negative controls (Table 17) or totally (Table 18).

There is no clearly documented evidence of cryptosporidiosis or toxoplasmosis occurring from camping or hiking activities: however, sources of sporadic illness are often identified although not directly attributable to these pathogens (Boulware et al, 2003). Illnesses, nevertheless, could still occur simply because one oocyst is all that is needed to produce a coccidial infection (Lee and Winder, 1981). Therefore, without a total inactivation of oocysts, chemicals and treatment devices that were reportedly making several log reductions (Venczel et al. 1997) will not keep campers and hikers from danger.

Howard Backer recommended either heat or filtration in his review on field treatment of Cryptosporidium (1997). Although we did not test filtration devices, all water used in Example 6 was filtered through a 1μ filter. We would agree that heat is an excellent method to inactivate oocysts of coccidian parasites. We would add that the results from this study suggest that ultraviolet light also offers another effective protection against coccidian parasites in water for campers and hikers.

Chemicals/device. Six different purification chemicals and a water treatment device (Pristine [Water Purification System, Langley, B. C.], Micropur MP 1 water purification tablets [Katadyn® Products Inc., Wallisellen Switzerland], Coghlan's Drinking Water Tablets [Winnipeg, MB], MIOX® Purifier [Mountain Safety Research, Seattle, Wash.], Polar Pure Water Disinfectant [Polar Equipment Inc., Saratoga, Calif.], and a SteriPEN™ [Hydro-Photon, Inc., Blue Hill, Me.]) were used and within their expiration date, when specified. The doses of chemicals or use of the device were in accordance to the manufacturer's instructions but scaled down to treat 250 mL of water. The exception was the iodine products that were used at double the dosage recommended. Contact times were 4 hours for the Micropur tablets and MIOX® Purifier, 30 minutes for the Polar Pure tablets and Pristine® liquid, 40 minutes for Coghlan's tablets, and exposure to the ultraviolet light of the Steri-Pen™ was about 1 minute.

Bioassay. The bioassay was based on the sporulation of Eimeria acervulina oocysts. Unsporulated oocysts of Eimeria acervulina (obtained from Vetech Laboratories Inc., Guelph, Ontario where Eimeria acervulina oocysts are routinely worked on or harvested for manufacture to vaccine production) were counted in a haemocytometer under the microscope before the start of each experiment to determine the percentage that had sporulated. After exposing these unsporulated oocysts to treatments, oocysts were sporulated in 2.5% potassium dichromate, at 29.5° C., for a minimum of 20 hours. Oocysts only sporulate if they are living and will appear with 4 well defined sporocysts within each oocyst, distinct from the undifferentiated or unsporulated oocysts. However, oocysts can remain unsporulated as the result of inactivation by treatments or already damaged or dead prior to treatments. Therefore, the only valid criterium is by comparing the sporulation rate of the treated with the untreated control. At least 100 oocysts for each treatment were counted

In Experiment 1, unsporulated oocysts were collected from the faeces of chicks shed overnight. But for the remaining experiments unsporulated oocysts were harvested directly from the duodenum of chicks. In Experiment 1, about 2.0×10⁶ unsporulated oocysts were used in 250 mL of tap water for testing each chemical or device, and in Experiment 2 about 1.0×10⁵ oocysts in 250 mL of tap water were used to test each chemical or device.

Methodology. Oocysts to be treated were first added to 250 mL of municipal tap water (groundwater source, filtered through a 1μ filter) in 500 mL flasks. The chemical treatments were then added and the suspension was briefly shaken before standing at room temperature (20-22.5° C.) over the prescribed contact time. Oocysts from each 250 mL of treated water were spun at 600×g for 5 minutes. Pellets were pooled to a total volume of about 2 mL in two, 15 mL test tubes and sporulated in 2.5% potassium dichromate solution in a shaking water bath at 29.5° C. for at least 20 hours. For testing the ultraviolet light, oocysts were added to a water bottle (dimensions 195 mm high, 205 mm circumference) and the bottle was inverted and shaken during the ultraviolet light exposure of about 1 min. For each experiment a positive control was also prepared from untreated and unsporulated oocysts and a negative control which was placed in a 50 mL centrifuge tube with the sample submerged below hot water (77-80° C. water) for 30 min.

Turbidity measurements were made in a Monitek, turbidometer, model TA1 standardized with a sample of 10 NTU.

Results. Results are presented in Tables 17 and 18. Sporulation rates of oocysts exposed to chemicals in Experiment 1 varied from 66% to 78%, similar to the 73% sporulation rate of the positive control (Table 17).

TABLE 17
The ability of unsporulated oocysts of Eimeria acervulina to
sporulate after exposure to water purification chemicals or
an ultraviolet light device. Water was spiked with 2 × 10⁶
unsporulated oocysts/250 mL tap water before treatment.

| | | Experiment 1 | |
|---|---|---|---|
| | | Sporulated | |
| | Disinfectant | oocysts/total | Sporulation |
| Product name/control | type | oocysts | rate |
| Heated control | | 4/100 | 4.0% |
| (negative control) | | | |
| Positive control | | 207/282 | 73% |
| Coghlan's | Iodine | 179/271 | 66% |
| Polar Pure | | 160/237 | 68% |
| Micropur MP1 | Chlorine | 141/182 | 78% |
| Pristine ® | dioxide | 149/204 | 73% |
| MIOX ® | Mixed oxidants | 241/316 | 76% |
| Steri-Pen ™ | Ultraviolet | 19/500 | 3.8% |
| | light | | |

It appeared that there was some sporulation of oocysts exposed to the ultraviolet light device (3.8%), but this was no higher than the negative control at 4%. This small amount of sporulation, in the negative control, likely took place during overnight fecal collection rather than due to the treatment. Unsporulated oocysts collected directly from the duodenum did not show this phenomenon. Therefore the rest of the experiments were tested on oocysts collected directly from excised duodenum.

In Experiment 2, using fewer numbers of unsporulated oocysts to spike the water, all treatments performed better, with much lower rates of sporulation (Table 18).

TABLE 18
The ability of unsporulated oocysts of Eimeria acervulina to
sporulate after exposure to water purification chemicals or
an ultraviolet light device. Water was spiked with 1 × 10⁵
unsporulated oocysts/250 mL tap water before treatment.

| | | Experiment 2 | |
|---|---|---|---|
| | | Sporulated | |
| | Disinfectant | oocysts/total | Sporulation |
| Product name/control | type | oocysts | rate |
| Heated control | | 0/361 | 0% |
| (negative control) | | | |
| Positive control | | 253/406 | 62% |
| Coghlan's | Iodine | 74/148 | 50% |
| Polar Pure | | 65/105 | 62% |
| Micropur MP1 | Chlorine | 95/536 | 18% |
| Pristine^{®} | dioxide | 126/486 | 26% |
| MIOX ® | Mixed oxidants | 50/101 | 50% |
| Steri-Pen ™ | Ultraviolet | 0/285 | 0% |
| | light | | |

The turbidity of the spiked water in Experiment 1 was about 0.88 NTU, the water appearing cloudy. Turbidity was measured at 0.50 NTU for the spiked water in Experiment 2, the water appearing clear to the naked eye.

### EXAMPLE 7

### Testing the Effect of Liquid Disinfectants on Unsporulated Oocysts of Eimeria acervulina

TABLE 19
Effect of liquid disinfectants on unsporulated
oocysts of Eimeria acervulina.

| | Trial 1 | Trial 2 |
|---|---|---|
| Oocysts/treatments^{a} | Sporulation rates (%)^{b} | |
| No treatment | 78 | 89 |
| 25% hydrogen peroxide, 5% | 31 | 9 |
| peracetic acid | | |
| 12.5% hydrogen peroxide, 2.5% | 41 | |
| peracetic acid | | |
| 2.5% hydrogen peroxide, 0.5% | | 74 |
| peracetic acid | | |
| 7.5% hydrogen peroxide | | 74 |
| 2% hydrogen peroxide (Product A) | | 69 |
| Chlorine 600 ppm | 70 | |
| Chlorine 150 ppm | 89 | |
| Heat treated | 0 | 0 |

^{a} 3 hours treatment
^{b} 29.5° C. shaking water bath for at least 20 hours

### LITERATURE CITED

Backer H. Water disinfection strategies for Cryptosporidium. Wilderness Environ Med. 1997 May; 8(2):75-7.
Boulware D R, Forgey W W, Martin W J II. Medical risks of wilderness hiking. Am J Med 2003; 114:288-293.
Gerba C P, Johnson D C, Hasan M N. Efficacy of iodine water purification tablets against Cryptosporidium oocysts and Giardia cysts. Wilderness Environ Med. 1997 May; 8(2):96-100.
Hrudey, S E and E J Hrudey. Safe drinking water: lessons from recent outbreaks in affluent nations/S E Hrudey and E J Hrudey. London: IWA Publishing, 2004.
Isaac-Renton J. Blatherwick J, Bowie W R, Fyfe M, Khan M, Li A, King A, McLean M, Medd L, Moorhead W, Ong C S, Robertson W. Epidemic and endemic seroprevalence of antibodies to Cryptosporidium and Giardia in residents of three communities with different drinking water supplies. Am J Trop Med Hyg. 1999 April; 60(4):578-83.
Lee E H, Winder N C. Eimeria acervulina infections from each of the four sporocysts of a single oocyst. Can J Comp Med 1981; 45(2):203-4.
Lee M B, Lee E H. Coccidial contamination of raspberries: mock contamination with Eimeria acervulina as a model for decontamination treatment studies. J Food Prot 2001; 64(11):1854-7.
Lee M B, Lee E H. Passage of a coccidial parasite (Eimeria acervulina) through the Eastern oyster (Crassostrea virginica). J Food Prot 2003; 66(4):679-81.
Quinn P J, Markey B K. Disinfection and disease prevention in veterinary medicine. In: Block S, ed. Disinfection, Sterilization, and Prevention, ed. 5. Philadelphia: Lippincott Williams & Wilkins; 2000:1069-1103
Report of the Commission of Inquiry into matters relating to the safety of the public drinking water in the City of North Battleford, Saskatchewan. Mar. 28, 2002. Honourable Justice Robert D. Laing, Commissioner. Retrieved on Nov. 8, 2005 from www.northbattlefordwaterinquiry.ca/final
Roach P D, Olson M E, Whitley G, Wallis P M. Waterborne Giardia cysts and Cryptosporidium oocysts in the Yukon, Canada. Appl Environ Microbiol. 1993 January; 59(1):67-73.
Stotish R L, Wang C C, Meyenhofer M. Structure and composition of the oocyst wall of Eimeria tenella. J. Parasitol. 1978; 64(6):1074-81.
Venczel L V, Arrowood M, Hurd M, Sobsey M D. Inactivation of Cryptosporidium parvum oocysts and Clostridium perfringens spores by a mixed-oxidant disinfectant and by free chlorine. Appl Environ Microbiol. 1997 April; 63(4):1598-601.
Wallis P M, Erlandsen S L, Isaac-Renton J L, Olson M E, Robertson W J, van Keulen H. Prevalence of Giardia cysts and Cryptosporidium oocysts and characterization of Giardia spp. isolated from drinking water in Canada. Appl Environ Microbiol. 1996 August; 62(8):2789-97.

1. A device for testing the efficacy of a disinfectant treatment comprising
(a) a culture of live unsporulated free-living protozoan parasites and
(b) optionally, a receptacle for facilitating contacting of the disinfectant treatment with the culture.

2. The device of claim 1 wherein the culture of live unsporulated free-living protozoan parasites is divided into
(i) a first culture for testing with the disinfectant treatment;
(ii) a second culture to be stored for comparison with (i).

3. The device of claim 2 wherein the device further comprises
(a) (iii) a third culture heated to 80° C. for testing with a disinfectant treatment as a control group.

4. The device of claim 1 further comprising
(c) information on the predicted sporulation rates according to the age of the culture and storage conditions.

5. The device of claim 1 wherein the culture, the first culture, the second culture and the third culture are stored under conditions that prevent sporulation prior to the testing with the disinfectant treatment.

6. The device of claim 1 wherein the storage conditions include a temperature of from 2° C. to 7° C.

7. The device of claim 1 wherein the storage conditions include a temperature of about 4° C.

8. The device of claim 1 wherein the parasite is an Apicomplexa.

9. The device of claim 8 wherein the Apicomplexa is a coccidian parasite.

10. The device of claim 9 wherein the coccidian parasite is Eimeria acervulina.

11. The device of claim 9 wherein the coccidian parasite is Eimeria tenella.

12. The device of claim 1 wherein the device is stored for up to 30 days.

13. A method for testing the efficacy of a disinfectant treatment comprising
(a) contacting a culture of live unsporulated protozoan parasites in the free-living stage with an effective amount of the disinfectant treatment, and
(b) determining the percentage of protozoan parasites that have survived,
wherein the culture of (a) is stored under conditions that prevent sporulation prior to the contacting.

14. A method for testing the efficacy of a disinfectant treatment comprising steps in the following order:
(a) obtaining a stock culture of live protozoan parasite oocysts in the free-living stage containing a known and substantial quantity of live unsporulated oocysts and a known quantity of sporulated oocysts wherein the stock culture is stored under conditions that prevent sporulation;
(b) determining the ratio of the sporulated oocysts to the total number of oocysts in the stock culture of oocysts;
(c) exposing the stock culture of oocysts to the disinfectant treatment;
(d) exposing the thus treated culture of oocysts to sporulation inducing conditions;
(e) determining the number of unsporulated oocysts and sporulated oocysts in the treated culture;
(f) determining the ratio of the sporulated oocysts to the total number of oocysts in the treated culture of oocysts; and
(g) determining the effectiveness of the disinfectant treatment by comparing the ratio determined in step (b) with the ratio determined in step (f) and calculating the sporulation rate.

15. The method of claim 13 further comprising a second culture of oocysts that is the same as the stock culture in (a) and which is exposed to the same steps except it does not contact nor is exposed to the disinfectant treatment, thereby acting as a control.

16. The method of claim 15 further comprising a second culture of oocysts that is the same as the stock culture in (a) and which is exposed to the same steps except it does not contact nor is exposed to the disinfectant treatment, thereby acting as a control.

17. The method of claim 13 wherein the parasite is Apicomplexa.

18. The method of claim 17 wherein the Apicomplexa is a coccidian parasite.

19. The method of claim 18 wherein the coccidian parasite is Eimeria acervulina.

20. The method of claim 18 wherein the coccidian parasite is Eimeria tenella.
